(19) United States
(12) Patent Application Publication
(10) Pub. No.: US 2016/0038176 A1
(43) Pub. Date: Feb. 11, 2016

(54) Lever Locking Grip

(71) Applicant: Walter Smith, St. Petersburg, FL (US)

(72) Inventor: Walter Smith, St. Petersburg, FL (US)

(21) Appl. No.: 14/455,989

(22) Filed: Aug. 11, 2014

(51) Int. Cl. A61B 17/34; A61M 37/00

(52) U.S. Cl. CPC A61B 17/34; A61M 37/0076; A61B 2017/347; A61B 2017/0023

(57) ABSTRACT

A grip for removably supporting a needle holder includes a base portion that has a longitudinal channel sized for accepting a needle holder. The grip has a lever pivotally attached to the base portion. The lever has a lobe that interferes with the longitudinal channel when the lever is in a closed position and the lobe is positioned away from the channel when the lever is rotated into an open position. The lobe holds the needle holder within the longitudinal channel while the lever is in the closed position and the needle holder is released while the lever is in the open position.

### FIELD

This invention relates to the field of body marking and modification and more particularly to a system for releasably holding a needle cartridge used in body marking.

### BACKGROUND

Body marking by inserting pigmentation under the dermis layer of the skin has been known to have been performed for many years. Although there are many forms of such marking, such as permanent makeup, the word “tattoo” is often used to describe the insertion of inks beneath the dermis layer of one's skin. The word “tattoo” is believed to have evolved from the Polynesian word tatau or the Tahitian word tatu, as noted in the logs of explorer James Cook.

The art of body marking has progressed from those early days and now, there are many small operations that practice the art of tattooing and permanent makeup.

Being that such body marking requires an ink to be inserted beneath the dermis layer, it is too easy to pass various infectious pathogens from one recipient of body marking to another or to the person who is making the tattoo or applying the permanent makeup, etc. There are now, in most parts of the world, many procedures for providing for sterilization of needles and other equipment used to insert pigments beneath the dermis layer of a recipient, including the use of disposable needles, high temperature/steam sterilization of equipment and devices used to insert the pigments, disposable components, etc.

One area of sterilization that has been difficult is the disposal or sterilization of the needle cartridge, which is generally an elongated, typically cylindrical hollow device that is lodged within a grip and held within the grip by a set screw. In current systems, after one or more pigments are inserted beneath the dermis layer of a recipient, the needle cartridge is released from the grip by loosening of the set screw and allowing the needle cartridge to slide out of the grip, either into a hazardous material storage bin for disposable needle cartridges, or into a sterilization system for reusable needle cartridges. The process of loosening of one or more set screws on the grip adds risk to the process. There is unneeded handling of the grip, the screwdriver is a sharp object and, especially when fluids are present, it is possible for the tip of the screwdriver to slip off the set screw and potentially pierce the skin of the practitioner, and the screwdriver adds one more instrument that has the potential of transferring pathogens from one recipient to another.

What is needed is a grip that will improve the process of removing the needle cartridge from the grip without the need for an additional tool.

### SUMMARY

In one embodiment, a grip for removably supporting a needle holder is disclosed. The grip includes a base portion that has a longitudinal channel sized for accepting a needle holder. The grip also has a lever pivotally attached to the base portion. The lever has a lobe that interferes with the longitudinal channel when the lever is in a closed position and the lobe is positioned away from the channel when the lever is rotated into an open position. The lobe holds the needle holder within the longitudinal channel while the lever is in the closed position and the needle holder is released while the lever is in the open position.

In another embodiment, a method of using a grip with a needle holder is disclosed. The method includes providing a needle holder and a grip. The grip having a base portion and a lever; the base portion has a longitudinal channel that is sized for accepting the needle holder. The lever is pivotally attached to the base portion and has a lobe that interferes with the longitudinal channel when the lever is in a closed position and is positioned away from the channel when the lever is rotated into an open position. The method includes rotating the lever into the open position then sliding the needle holder into the longitudinal channel then rotating the lever into the closed position, thereby retaining the needle holder within the longitudinal channel. After such, skin art is created by an artist with the aid of the grip and needle holder. When finished creating art, the artist rotates the lever into the open position, thereby releasing the needle holder for disposal or sterilization.

In another embodiment, a grip that removably supports a needle holder is disclosed. The grip includes a base portion with a longitudinal channel that is sized for accepting a needle holder. The base portion has a feature for releasably maintaining the needle holder within the longitudinal channel. The feature for releasably maintaining is operated by an artist's finger, moving between an open position and a closed position. This feature for releasably maintaining retains the needle holder within the longitudinal channel while in the closed position and the needle holder is released when this feature for releasably maintaining is in the open position.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a perspective view of a grip with a locking lever shown in the locked position.

FIG. 2 illustrates a perspective view of a grip with a locking lever shown in the unlocked position.

FIG. 3 illustrates an exploded view of a grip with a locking lever.

FIG. 4 illustrates a cut-away view of a grip with a locking lever.

FIG. 5 illustrates a side plan view of a locking lever.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

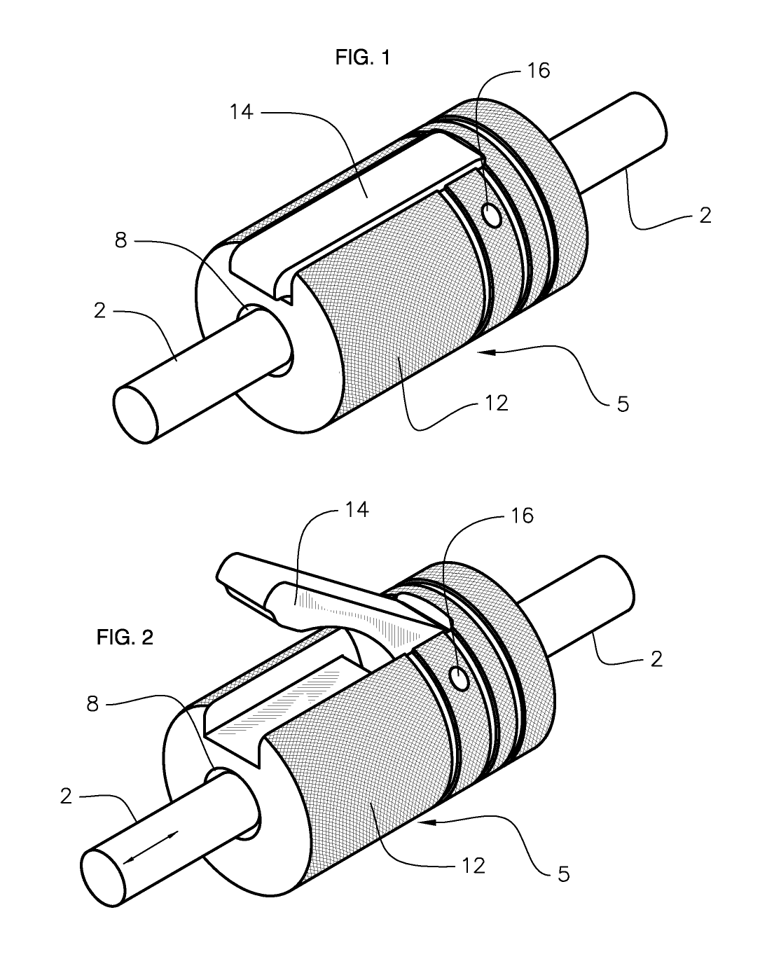

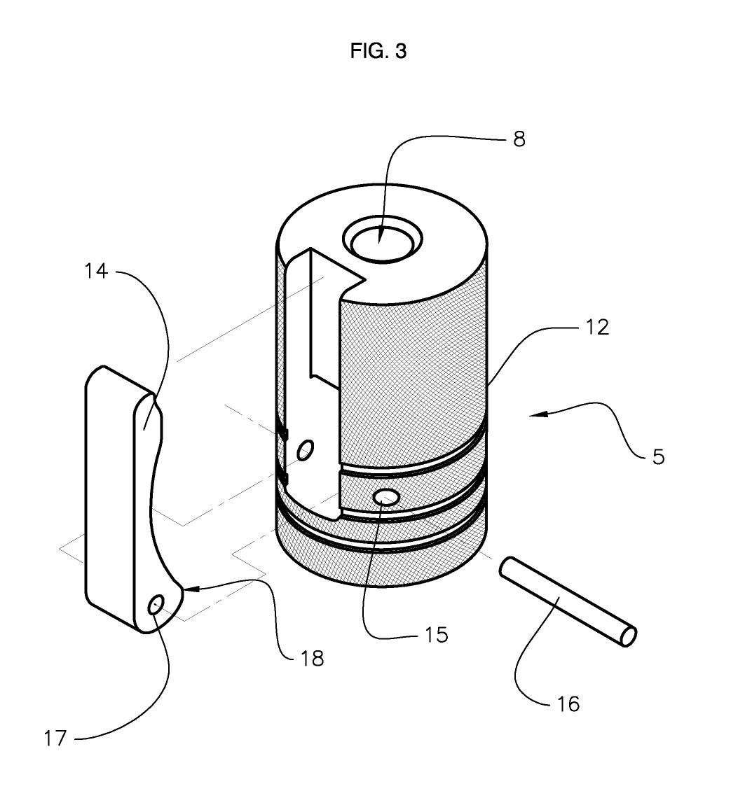

Referring to FIGS. 1 and 2, perspective views of a grip 5 with a locking lever 14 are shown in the locked position (FIG. 1) and in the unlocked position (FIG. 2). The base portion 12 of the grip 5, preferably has a textured surface, though not required, to help hold the grip 5, especially when the grip becomes wet. The needle holder 2 is shown inserted within an elongated, longitudinal channel 8 of the grip 5.

The grip includes a lock and release mechanism. In the locked position, the lock and release mechanism maintains the needle holder 2 within the elongated, longitudinal channel 8 while in the unlocked position, the lock and release mechanism allows removal of the needle holder 2 from the elongated, longitudinal channel 8, preferably with little or no force other than gravity. The lock and release mechanism does not require tools and did prior grip devices having set screws, etc., and the lock and release mechanism operates by the artist's fingers alone.

The lock and release mechanism is shown as a lever 14 that is rotatably interfaced to the base portion 12 by a pivot pin 16. In such, lock and release mechanism (lever 14) has an open position (FIG. 2) in which the lobe 18 is not positioned directly between the pivot pin 16, and therefore, not exerting pressure on the needle holder 2 within the longitudinal channel 8. The lock and release mechanism (lever 14) has an closed position (FIG. 1) in which the lobe 18 is positioned directly between the pivot pin 16, and therefore, exerts pressure on the needle holder 2 within the longitudinal channel 8, maintaining the needle holder 2 in place.

Although there are many ways anticipated to hold a lever 14 to the base portion 12 of the grip 5, the way shown includes a pivot pin 16. In other embodiments, other rotatable connection systems are anticipated such as screws, shafts, nubs on the lever 14 that mate with detents in the base portion 12, nubs in the base portion 12 that mate with detents in the lever 14, etc. Any rotatable connection system is anticipated.

In some embodiments, the lock and release mechanism is other than a lever and has a momentary operation, in that, after exerting pressure to move the lock and release mechanism into the unlock position, the lock and release mechanism automatically reverts back into the locked position, for example by resilient force or magnetic force. For example, a sliding button is slideably interfaced to the base portion 12 and biased in one direction by a spring, being the locked position in which the sliding button exerts pressure against any needle holder 2 within the elongated channel 8. Upon exerting pressure on the sliding button in a direction that counteracts the spring, the sliding button moves and disengages with the needle holder 2 that is within the channel 8, thereby releasing the needle holder 2 from the channel 8.

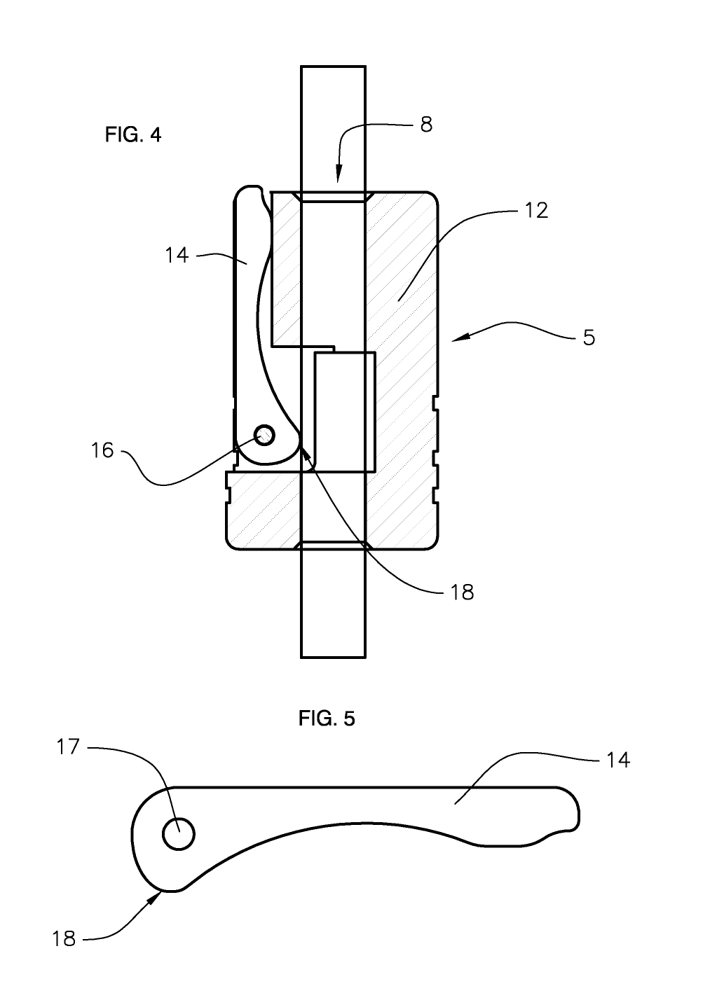

As shown in FIG. 1, the lever 14 is flush with the base portion 12 of the grip 5. In this position, as is shown in FIG. 4, a lobe 18 of the lever 14 impedes within the channel 8, such that, when a needle holder 2 is within the channel 8, the lobe 18 holds the needle holder 2 within the channel 8. When the lever 14 is rotated into the open position as shown in FIG. 2, the lobe 18 disengages with any needle holder 2 present within the channel 8, thereby allowing the needle holder 2 to slide out of the channel 8 with little or no external force being exerted.

After use of the needle holder 2, it is possible that bodily fluids such as blood have contaminated the needle holder 2. Therefore, the needle holder 2 must be either disposed (e.g. for plastic, disposable needle holders 2) or sterilized (e.g. for metal, reusable needle holders 2). To dispose of a contaminated needle holder 2, the artist holds the grip 5 in a substantially vertical orientation above either a hazardous material bin (for disposable needle holders 2) or over a sterilization device/bath (for reusable needle holders 2) and moves the lever 14 into the open position (as shown in FIG. 2), at which time the lob 18 rotates away from the needle holder 2 and the needle holder 2 moves freely out of the grip 5 and into either the hazardous material bin or the sterilization device/bath. A new or already sterilized needle holder 2 is then inserted into the grip 5 and the lever 14 is moved into the closed position (as shown in FIG. 1), thereby the lobe 18 applies pressure onto the needle holder 2 keeping the needle holder 2 in place.

Referring to FIG. 3, an exploded view of a grip 5 with a locking lever 14 is shown. In this view, the assembly of an exemplary grip 5 is shown with a pivot pin 16 that passes through holes 15 in the base portion 12 of the grip 5 and through a hole 17 in the lever 14. Although there are many ways anticipated to hold a lever 14 to the base portion 12 of the grip 5, the way shown includes a pivot pin 16 that passes through holes 15 in the base portion 12 of the grip 5 and through a hole 17 in the lever 14, thereby allowing rotation of the lever 14 to/from the closed, locked position (as in FIG. 1) and the open, unlocked position (as in FIG. 2). In other embodiments, other rotatable connection systems are anticipated such as screws, shafts, nubs on the lever 14 that mate with detents in the base portion 12, nubs in the base portion 12 that mate with detents in the lever 14, etc. Any rotatable connection system is anticipated.

The exemplary lever 14 has a lobe 18 that applies pressure to the needle holder 2 when in the closed position (see FIG. 1) to secure the needle holder 2 within the channel 8. When the lever is moved to the open position (see FIG. 2), the lobe 18 disengages with the needle holder 2, allowing the needle holder 2 to fall out of the channel 8 or to be pushed or pulled out of the channel 8.

Referring to FIG. 4, a cut-away view of a grip 5 with a locking lever 14 is shown with the lever 14 in the closed position. In this view, the interaction between the cam 18 and the needle holder 2 within the channel 8 is visible. The lever 14 is shown in the closed position. In such, the cam 18 exerts force against the needle holder 2 which is positioned within the channel 8. When the lever 14 is moved into the open position (as in FIG. 2), the cam 18 rotates counterclockwise with respect to the orientation of FIG. 4, thereby relieving any force that was applied against the needle holder 2, allowing free movement of the needle holder 2 within the channel 8 and, therefore, easy removal of the needle holder 2 from the channel 8 for disposal or sterilization of the needle holder 2.

Referring to FIG. 5, a side plan view of a locking lever 14 is shown. The surface of the lobe 18 is further from the center of the pivot hole 17 than the surface of the lever 14 that is counter clockwise from the lobe 18. Therefore, when in the locked position, the lobe 18 interferes with movement of the needle holder 2 within the channel 8. When in the unlocked position (counter clockwise rotation with respect to FIG. 5), the thinner area of the lever 14 reduces contact pressure or disengages with the needle holder 2 within the channel 8, allowing for movement of the needle holder 2 up/down the channel 8 for removal (discard or sterilization) or for insertion of a new or sterilized needle holder 2.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A grip for removably supporting a needle holder, the grip comprising:
a base portion, the base portion having a longitudinal channel, the longitudinal channel sized for accepting a needle holder; and
a lever pivotally attached to the base portion, the lever having a lobe, the lobe interfering with the longitudinal channel when the lever is in a closed position and the lobe positioned away from the channel when the lever is rotated into an open position;
whereas the lobe holds the needle holder within the longitudinal channel while the lever is in the closed position and the needle holder is released while the lever is in the open position.

2. The grip of claim 1, wherein at least a portion of a surface of the base portion is textured.

3. The grip of claim 1, wherein the lever is pivotally attached to the base portion by a pivot pin.

4. The grip of claim 1, whereas the needle holder falls out of the longitudinal channel by force of gravity when the lever is in the open position.

5. The grip of claim 3, wherein the lobe is positioned directly between the pivot pin and the longitudinal channel when the lever is rotated into the closed position and the lobe is positioned further away from the longitudinal channel when the lever is rotated into the open position.

6. The grip of claim 1, wherein the longitudinal channel is cylindrical.

7. A method of using a grip with a needle holder, the method comprising:
providing a needle holder;
providing a grip having a base portion and a lever; the base portion having a longitudinal channel, the longitudinal channel sized for accepting the needle holder; and the lever being pivotally attached to the base portion; the lever having a lobe, the lobe interfering with the longitudinal channel when the lever is in a closed position and the lobe positioned away from the channel when the lever is rotated into an open position;
rotating the lever into the open position;
sliding the needle holder into the longitudinal channel;
rotating the lever into the closed position, thereby retaining the needle holder within the longitudinal channel;
creating skin art with the aid of the grip and needle holder; and
rotating the lever into the open position, thereby releasing the needle holder for disposal or sterilization.

8. The method of claim 7, wherein at least a portion of a surface of the base portion of the grip is textured.

9. The method claim 7, wherein the lever is pivotally attached to the base portion by a pivot pin.

10. The method claim 7, whereas in the step of releasing the needle holder for disposal or sterilization, the needle holder falls out of the longitudinal channel by force of gravity.

11. The method claim 9, wherein the lobe is positioned directly between the pivot pin and the longitudinal channel of the grip when the lever is rotated into the closed position and the lobe is positioned further away from the longitudinal channel when the lever is rotated into the open position.
The method claim 7, wherein the longitudinal channel of the base portion of the grip is cylindrical.

12. A grip for removably supporting a needle holder, the grip comprising:
a base portion, the base portion having a longitudinal channel, the longitudinal channel sized for accepting a needle holder; and
means for releasably maintaining the needle holder within the longitudinal channel;
whereas the means for releasably maintaining is operated by an artist's finger between an open position and a closed position, whereas the means for releasably maintaining retains the needle holder within the longitudinal channel while the means for releasably maintaining is in the closed position and the needle holder is released when the means for releasably maintaining is in the open position.

13. The grip of claim 12, wherein at least a portion of a surface of the base portion is textured.

14. The grip of claim 12, wherein the means for releasably maintaining comprises a lever.

15. The grip of claim 14, wherein the lever is pivotally attached to the base portion by a pivot pin.

16. The grip of claim 12, whereas the needle holder falls out of the longitudinal channel by force of gravity when the means for releasably maintaining is in the open position.

17. The grip of claim 15, wherein the lever comprises a lobe that is positioned directly between the pivot pin and the longitudinal channel when the lever is rotated into the closed position and the lobe is positioned further away from the longitudinal channel when the lever is rotated into the open position.

18. The grip of claim 12, wherein the longitudinal channel is cylindrical.
